(19) United States
(12) Patent Application Publication
(10) Pub. No.: US 2026/0199880 A1
(43) Pub. Date: Jul. 16, 2026

(54) CATALYST FOR THE PRODUCTION OF METHYL METHACRYLATE

(71) Applicants: Dow Global Technologies LLC, Midland, MI (US); Rohm and Haas Company, Collegeville, PA (US)

(72) Inventors: Kirk W. Limbach, Dresher, PA (US); Victor J. Sussman, Midland, MI (US); Alexey Kirilin, Terneuzen (NL); Wen-Sheng Lee, Midland, MI (US)

(21) Appl. No.: 19/125,366

(22) PCT Filed: Nov. 20, 2023
(86) PCT No.: PCT/US2023/080437

§ 371 Date: Apr. 29, 2025

Related U.S. Application Data
(60) Provisional application No. 63/431,035, filed on Dec. 8, 2022.

(51) Int. Cl. B01J 23/78; B01J 21/08; B01J 23/02; B01J 27/224; B01J 37/00; B01J 37/02; B01J 37/03; B01J 37/04; B01J 37/08; C07C 67/44

(52) U.S. Cl. CPC B01J 23/78; B01J 21/08; B01J 23/02; B01J 27/224; B01J 37/009; B01J 37/0201; B01J 37/0236; B01J 37/035; B01J 37/04; B01J 37/088; C07C 67/44

(57) ABSTRACT

A catalyst for oxidative esterification of methacrolein to methyl methacrylate comprises a support having an average diameter of at least 0.8 mm. The support is selected from an oxide of silicon, a carbide of silicon, a metal oxide, and a metal carbide. The catalyst further comprises nickel oxide and gold particles disposed on the support. The gold particles and have an average diameter of less than 12 nm and a standard deviation of +/−4 nm. A method for preparing methyl methacrylate from methacrolein and methanol using the catalyst is also disclosed.

### BACKGROUND OF THE INVENTION

The invention relates to a catalyst and method for preparing methyl methacrylate from methacrolein and methanol.

Heterogeneous catalysts for use in producing carboxylic esters, including methyl methacrylate, from aldehydes are known.

U.S. Pat. No. 8,461,373 discloses a catalyst comprising oxidized nickel and at least one element selected from nickel, palladium, platinum, ruthenium, gold, silver, and copper. The diameter of the catalyst ranges from 10 to 200 μm.

WO 2016/113106 discloses a catalyst comprising gold, silicon oxide, aluminum oxide, and an oxide of at least one element selected from alkali metals, alkaline earth metals, lanthanides having atomic numbers from 57 to 71, Y, Sc, Ti, Zr, Cu, Mn, Pb and Bi. The mean diameter of the catalyst ranges from 10 to 200 μm.

In the production of carboxylic esters from aldehydes, two types of reactors are most often used. Slurry or continuously stirred tank reactors use a small catalyst (<200 μm) that is kept in suspension, whereas fixed bed reactors use a large catalyst (>200 μm) that is fixed in position within the reactor. Due to the difference in reactor designs, catalysts suitable for one type of reactor are not suitable for another type of reactor. Each type of reactor subjects the catalyst to different conditions, including the forces to which the catalyst is exposed, and the catalysts are designed differently based on the respective size of the catalysts in which the ratio of catalytically active area to the overall volume of the catalyst is drastically different.

However, there is a need for an improved catalyst and process for production of methyl methacrylate that is effective and active for longer lifespans.

### SUMMARY OF THE INVENTION

One aspect of the present invention relates to a catalyst for oxidative esterification of methacrolein to methyl methacrylate comprises a support having an average diameter of at least 0.8 mm. The support is selected from an oxide of silicon, a carbide of silicon, a metal oxide, and a metal carbide. The catalyst further comprises gold particles and nickel oxide disposed on the support. The gold particles and have an average diameter of less than 12 nm and a standard deviation of +/−4 nm.

Another aspect of the present invention relates to a method for preparing methyl methacrylate from methacrolein and methanol; said method comprising contacting in a reactor a mixture comprising methacrolein, methanol and oxygen in the presence of a catalyst comprising a support with nickel oxide and gold particles disposed on the support. The support has an average diameter of at least 0.8 mm and is selected from an oxide of silicon, a carbide of silicon, a metal oxide, and a metal carbide. The catalyst further comprises nickel oxide and gold particles disposed on the support. The gold particles and have an average diameter of less than 12 nm and a standard deviation of +/−4 nm.

### DETAILED DESCRIPTION OF THE INVENTION

All percentage compositions are weight percentages (wt %), and all temperatures are in ° C., unless otherwise indicated. Averages are arithmetic averages unless otherwise indicated. The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters. Unless otherwise stated, the average diameter of a particle refers to the average diameter of the particle after the catalyst has been prepared and before the catalyst has been used. An aged catalyst is a catalyst that has been used.

The catalyst of the present invention comprises a support with gold particles and nickel oxide disposed on the support.

The support has an average diameter of at least 0.8 mm. More preferably, the support has an average diameter of at least 1.2 mm, even more preferably at least 1.5 mm, and still more preferably at least 2 mm.

The support comprises a material selected from an oxide of silicon, a carbide of silicon, a metal oxide, and a metal carbide. The metal of the metal oxide and the metal carbide may be selected from, for example, aluminum, titanium, zirconium, zinc, and magnesium. Preferably, the support is capable of withstanding long-term use in an oxidative esterification reactor. Materials that are capable of withstanding prolonged use are able to avoid being crushed or pulverized during use.

The support material may further comprise at least one metal oxide where the metal is selected from aluminum, titanium, lanthanides, zirconium, alkali metals, alkaline earth metals, nickel, cobalt, zinc, magnesium, tellurium, antimony, and bismuth. The at least one metal oxide may be used to modify support material.

Preferably, the support comprises, consists of, or consists essentially of an oxide of silicon. More preferably, the support comprises, consists of, or consists essentially of an oxide of silicon modified with titanium oxide. As used herein with respect to the support, the phrase “consists essentially of” excludes the presence of materials that would degrade the mechanical strength of the support. Alternatively, “consists essentially of” means that the support comprises at least 95 wt % of the stated material with respect to the total weight of the support.

Preferably, the support has a surface area greater than 10 m²/g, preferably greater than 30 m²/g, preferably greater than 50 m²/g, preferably greater than 100 m²/g, preferably greater than 120 m²/g.

Preferably, the aspect ratio of the catalyst particle is no more than 10:1, preferably no more than 5:1, and preferably no more than 3:1. Although the shape is not limited, preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

The nickel oxide and gold particles are preferably disposed on an outer surface of a support material. Preferably, at least 75% by weight of the gold particles are within an outer 25% of the volume of the catalyst. More preferably, at least 80%, still more preferably at least 85%, by weight of the gold particles are within an outer 25% of the volume of the catalyst.

The nickel oxide disposed on the support is preferably in the form of nanoparticles.

Preferably, at least 75% of the gold particles by number of gold particles are within at least 20 nm of nickel oxide, such as within at least 20 nm of a nickel oxide nanoparticle when the nickel oxide is in the form of nanoparticles. As used herein, the phrase “within at least X nm” means that an edge of a gold particle is within X nm of the nickel oxide, e.g., within X nm of an edge of a nickel oxide nanoparticle nearest the gold particle. Preferably, at least 75% of the gold particles are within at least 15 nm of nickel oxide, more preferably within at least 12 nm of nickel oxide, and even more preferably within at least 10 nm of nickel oxide.

More preferably, the nickel oxide is in the form of nanoparticles, and at least 75% of the gold particles by number of the gold particles are within at least 20 nm of two nickel oxide nanoparticles, i.e., an edge of the gold particle is within at least 20 nm of an edge of the two nickel oxide nanoparticles nearest the gold particle. Preferably, at least 75% of the gold particles are within at least 15 nm of two nickel oxide nanoparticles, more preferably within at least 12 nm of two nickel oxide nanoparticles, and even more preferably within at least 10 nm of two nickel oxide nanoparticles.

Even more preferably, the nickel is in the form of nanoparticles, and at least 75% of the gold particles by number of the gold particles are within at least 20 nm of at least three nickel oxide nanoparticles, i.e., an edge of the gold particle is within at least 20 nm of an edge of at least the three nickel oxide nanoparticles nearest the gold particle. Preferably, at least 75% of the gold particles are within at least 15 nm of at least three nickel oxide nanoparticles, more preferably within at least 12 nm of at least three nickel oxide nanoparticles, and even more preferably within at least 10 nm of at least three nickel oxide nanoparticles.

The gold particles have an average diameter of less than 12 nm, preferably less than 10 nm, and more preferably less than 8 nm. The standard deviation of the average diameter of the gold particles is +/−4 nm, preferably +/−2.5 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x is the mean of the n number of particles, and n is at least 500.

Nickel oxide nanoparticles preferably have an average diameter of less than 5 times the average diameter of the gold particles, more preferably an average diameter of less than 4 times the average diameter of the gold particles, even more preferably an average particle diameter of less than 3 times the average diameter of the gold particles, still more preferably an average particle diameter of less than 2 times the average diameter of the gold particles, and yet more preferably an average particle diameter of less than 1.5 times the average diameter of the gold particles. Preferably, the nickel oxide nanoparticles have an average diameter at least the half the average diameter of the gold particles, and more preferably at least the same as the average diameter of the gold particles.

The amount by weight of the gold particles with respect to the amount of the nickel oxide may range from 1:1 to 1:20. Preferably, the weight ratio of gold particles to nickel oxide ranges from 1:2 to 1:15, more preferably, from 1:3 to 1:10, and even more preferably, from 1:3 to 1:6.

The amount by weight of the nickel oxide with respect to the amount by weight of the gold particles may range from 0.1:1 to 10:1, preferably from 0.2:1 to 5:1, more preferably from 0.33:1 to 3:1, and still more preferably from 0.5:1 to 2:1.

Preferably, the gold particles are evenly distributed among the nickel oxide. As used herein, the term “evenly distributed” means the gold particles are randomly dispersed among the nickel oxide with substantially no agglomeration of the gold particles, e.g., less than 10% by weight of the gold particles based on the total weight of the gold particles are in physical contact with another gold particle. Preferably, less than 7.5% by weight of the gold particles based on the total weight of the gold particles are in physical contact with another gold particle, and more preferably, less than 5% by weight of the gold particles based on the total weight of the gold particles are in physical contact with another gold particle.

Preferably, at least 75 wt % of the gold particles are in the outer 50% of the catalyst volume (i.e., the volume of an average catalyst particle), more preferably the outer 40% of catalyst volume, even more preferably the outer 30%, and still more preferably the outer 25. Preferably, the outer volume of any particle shape is calculated for a volume having a constant distance from its inner surface to its outer surface (the surface of the catalyst particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the gold particles are in the outer volume of the catalyst, preferably at least 97 wt %, preferably at least 99 wt %. Preferably, at least 90 wt % (preferably at least 95 wt %, preferably at least 97 wt %, preferably at least 99 wt %) of the gold particles are within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface. Preferably, the gold particles form an eggshell structure on the support particles. The eggshell layer may have a thickness of 500 microns or less, preferably 250 microns or less, and more preferably 100 microns or less.

Preferably, at least 0.1% by weight of the total weight of the gold particles are exposed on a surface of the catalyst. As used herein, the term “exposed” means that at least a portion of the gold particle is not covered by another gold particle or nickel oxide, i.e., the reactants can directly contact the gold particle. The gold particles may therefore be disposed within a pore of the support material and still be exposed by virtue of the reactant being able to directly contact the gold particle within the pore. More preferably, at least 0.25% by weight of the total weight of the gold particles are exposed on the surface of the catalyst, even more preferably, at least 0.5% by weight of the total weight of the gold particles are exposed on the surface of the catalyst, and still more preferably, at least 1% by weight of the total weight of the gold particles are exposed on the surface of the catalyst.

Preferably, the amount of gold as a percentage of the gold and the support is from 0.2 to 5 wt %, preferably at least 0.5 wt %, preferably at least 0.8 wt %, preferably at least 1 wt %, preferably at least 1.2 wt %; preferably no more than 4 wt %, preferably no more than 3 wt %, preferably no more than 2.5 wt %.

Preferably, the catalyst is produced by precipitating the gold and nickel from an aqueous solution of metal salts in the presence of the support. In one preferred embodiment, the catalyst is produced by an incipient wetness technique in which an aqueous solution of a suitable gold precursor salt and nickel salt is added to a porous inorganic oxide such that the pores are filled with the solution and the water is then removed by drying. The resulting material is then converted into a finished catalyst by calcination, reduction, or other pre-treatments known to those skilled in the art to decompose the gold salts and nickel salts into metals or metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. Especially preferred thiol compounds include thiomalic acid, 3-mercaptopropionic acid, thioglycolic acid, 2-mercaptoethanol and 1-thioglycerol, including their conjugate bases.

In one embodiment of the invention, the catalyst is produced by deposition precipitation in which a porous inorganic oxide is immersed in an aqueous solution containing a suitable gold precursor salt and a nickel salt and is the salts are then made to interact with the surface of the inorganic oxide by adjusting the pH of the solution. The resulting treated solid is then recovered (e.g. by filtration) and then converted into a finished catalyst by calcination, reduction, or other pre-treatments known to those skilled in the art to decompose the gold salts and nickel salts into metals or metal oxides.

Preferably, the process for producing methyl methacrylate (MMA) is performed in an oxidative esterification reactor (OER). The catalyst particles may be present in a slurry or in a catalyst bed, preferably a catalyst bed. The catalyst particles in the catalyst bed typically are held in place by solid walls and by screens or catalyst support grids. In some configurations, the screens or grids are on opposite ends of the catalyst bed and the solid walls are on the side(s), although in some configurations the catalyst bed may be enclosed entirely by screens. Preferred shapes for the catalyst bed include a cylinder, a rectangular solid and a cylindrical shell; preferably a cylinder. The OER further comprises a liquid phase comprising methacrolein, methanol and MMA and a gaseous phase comprising oxygen. The liquid phase may further comprise byproducts, e.g., methacrolein dimethyl acetal (MDA) and methyl isobutyrate (MIB). Preferably, the liquid phase is at a temperature from 40 to 120° C.; preferably at least 50° C., preferably at least 60° C.; preferably no more than 110° C., preferably no more than 100° C. Preferably, the catalyst bed is at a pressure from 0 to 2000 psig (101 kPa to 14 MPa); preferably no more than 2000 kPa, preferably no more than 1500 kPa.

The OER typically produces MMA, along with methacrylic acid and unreacted methanol. Preferably, methanol and methacrolein are fed to the reactor in a methanol:methacrolein molar ratio from 1:10 to 100:1, preferably from 1:2 to 20:1, preferably from 1:1 to 10:1. Preferably, a catalyst bed further comprises inert or acidic materials above and/or below the catalyst. Preferred inert or acidic materials include, e.g., alumina, clay, glass, silica carbide and quartz. Preferably, the inert or acidic material has an average diameter equal to or greater than that of the catalyst, preferably no greater than 20 mm. Preferably, the reaction products are fed to a methanol recovery distillation column which provides an overhead stream rich in methanol and methacrolein; preferably this stream is recycled back to the OER. The bottoms stream from the methanol recovery distillation column comprises MMA, MDA, methacrylic acid, salts and water. In one embodiment of the invention, MDA is hydrolyzed in a medium comprising MMA, MDA, methacrylic acid, salts and water. MDA may be hydrolyzed in the bottoms stream from a methanol recovery distillation column; said stream comprising MMA, MDA, methacrylic acid, salts and water. In another embodiment, MDA is hydrolyzed in an organic phase separated from the methanol recovery bottoms stream. It may be necessary to add water to the organic phase to ensure that there is sufficient water for the MDA hydrolysis; these amounts may be determined easily from the composition of the organic phase. The product of the MDA hydrolysis reactor is phase separated and the organic phase passes through one or more distillation columns to produce MMA product and light and/or heavy byproducts. In another embodiment, hydrolysis could be conducted within the distillation column itself.

One preferred embodiment is a recycle reactor with cooling capacity in the recycle loop. Another preferred embodiment is a series of reactors with cooling and mixing capacity between the reactors.

Preferably, oxygen concentration at a reactor outlet is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the reactor. Preferably, the oxygen concentration in a gas stream exiting the reactor is no more than 7.5 mol %, preferably no more than 7.25 mol %, preferably no more than 7 mol %, based on the total amount of the gas stream exiting the reactor.

One preferred embodiment of the fixed bed reactor for oxidative esterification is a trickle bed reactor, which contains a fixed bed of catalyst and passes both the gas and liquid feeds through the reactor in the downward direction. In trickle flow, the gas phase is the continuous fluid phase. Thus, the zone at the top of the reactor, above the fixed bed, will be filled with a vapor phase mixture of nitrogen, oxygen, and the volatile liquid components at their respective vapor pressures. Under typical operating temperatures and pressures (50-90° C. and 60-300 psig (400-2000 kPa)), this vapor mixture is inside the flammable envelope if the gas feed is air. Thus, only an ignition source would be required to initiate a deflagration, which could lead to loss of primary containment and harm to the physical infrastructure and personnel in the vicinity. In order to address process safety considerations, a means to operate a trickle bed reactor while avoiding a flammable headspace atmosphere is operation with a gas feed containing a sufficiently low oxygen mole fraction to ensure the oxygen concentration in the vapor headspace is below the limiting oxygen concentration (LOC).

Knowledge of the LOC is required for the fuel mixture, temperature, and pressure of concern. Since the LOC decreases with increasing temperature and pressure, and given that methanol gives a lower LOC than the other two significant fuels (methacrolein and methyl methacrylate), a conservative design chooses a feed oxygen to nitrogen ratio that ensures a composition with less than the LOC at the highest expected operating temperature and pressure. For example, for a reactor operated at up to 100° C. and 275 psig (2 MPa), the feed oxygen concentration in nitrogen should not exceed 7.4 mol %.

### EXAMPLES

### Example #1 (Approximately 1.25 mm, Actual Size Varied from 0.85 To 1.70 mm, Au and Ni on Mg Modified SiO₂ Spheres)

To prepare the catalyst, a Mg modified SiO₂ support was first prepared as follows. Place 100 g of Cariact Q-10 SiO₂ in a container and treat it with a solution of magnesium nitrate hexahydrate in water prepared by dissolving 51 g of Mg(NO)₃·6H₂O in 100 mL of water and adding 5.5 g of 60% nitric acid. The suspension of silica was stirred with magnesium nitrate at 50° C. for 24 hours. The mixture was filtered and dried in a vacuum oven for 1 h at 80° C. and calcined overnight at 500° C.

The catalyst was then prepared by the following procedure. Suspend 50 g of Mg modified SiO₂ in 170 mL of deionized water. This slurry was heated to 90° C. while stirring until well dispersed. Separately, a solution was prepared containing 0.883 g of tetrachloroauric acid and 2.73 g nickel nitrate hexahydrate in 170 mL of deionized water. This solution was added over 30 minutes to the hot slurry while stirring. The mixture was allowed to stir for an additional 30 minutes and then the solid off was filtered off. The separated solid was washed three times with 100 mL portions of water, mixing it with the wash water for 5 minutes each time before filtering. The resulting solid was dried in air for 10 h at 105° C. and then calcined at 450° C. (5° C./min ramp) for 5 hours.

The resulting catalyst was 0.38 wt % Au, 0.57 wt % Ni, and 1.49 wt % Mg on a primarily SiO₂ support.

### Example #2 (3 mm Catalyst of Au on Ni Coated SiC Cylindrical Pellets)

A coated support was first prepared by impregnation-evaporation method. 10 g of SiC 3 mm cylindrical extrudates were loaded in a round bottom flask followed by 11 ml of 1M Nickel (II) nitrate hexahydrate, Ni(NO3)2*6H2O) solution, in deionized water. The flask was placed on a RotaVap. Water was removed under vacuum at continuous flask rotation at 45-50° C. The as-prepared support was dried under vacuum for 30 min at 45-50° C. and then calcined on air in the box oven using the following procedure: room temperature to 120° C. at 3° C./min, hold for 2 h, 120 to 600° C. at 2° C./min, hold for 4 h, cool down to room temperature in 2 h. The as-promoted SiC support contains ~5.1 wt % of Ni (in the form of oxide). Ni—SiC support had a BET surface area of 28 m²/g and avg. pore width of 12.5 nm.

Sodium aurothiomalate (I) was then utilized to put gold, Au, on the catalyst support by first preparing a stock solution of 0.1988 M sodium aurothiomalate (I) in deionized water. An impregnation solution was prepared by mixing 38.3 ml of the 0.1988 M sodium aurothiomalate (I) stock solution with 1.7 ml of deionized water until a transparent yellow solution was formed. The catalyst was prepared by incipient wetness impregnation followed by drying and calcination in air using a box oven equipped with air purge. 5 g of Ni—SiC support were impregnated with 2 ml of the impregnation solution dropwise until incipient wetness point. The impregnated material was dried and calcined in air in the box oven using the following procedure: room temperature to 120° C. at 5° C./min, hold for 1 h, 120 to 300° C. at 5° C./min, hold for 4 h, cool down to room temperature over 2 h.

The catalyst was shown to be active by running in a reactor to achieve high yield. The catalyst was 1.4 wt % Au and 5.1 wt % Ni on SiC support and achieved over 99% selectivity and a space-time yield of 21.8 mol MMA/Kg cat hr.

1. A catalyst for oxidative esterification of methacrolein to methyl methacrylate comprising:
a support having an average diameter of at least 0.8 mm, wherein the support comprises a material selected from the group consisting of an oxide of silicon, a carbide of silicon, a metal oxide, and a metal carbide;
nickel oxide disposed on the support; and
gold particles disposed on the support, wherein the gold particles have an average diameter of less than 12 nm and a standard deviation of +/−4 nm.

2. The catalyst of claim 1, wherein the nickel oxide is in the form of nanoparticles.

3. The catalyst of claim 1, wherein at least 75% of the gold particles are positioned within 20 nm of the nickel oxide.

4. The catalyst of claim 1, wherein the gold particles are evenly distributed among the nickel oxide.

5. The catalyst of claim 1, wherein the support further comprises at least one metal oxide where the metal is selected from the group consisting of aluminum, titanium, lanthanides, zirconium, alkali metals, alkaline earth metals, nickel, cobalt, zinc, magnesium, tellurium, antimony, and bismuth.

6. The catalyst of claim 1, wherein the gold particles have an average diameter of less than 10 nm and a standard deviation of +/−2.5 nm.

7. The catalyst of claim 1, wherein at least 0.1% by weight of the total weight of the gold particles are exposed on a surface of the catalyst.

8. The catalyst of claim 7, wherein at least 0.5% by weight of the total weight of gold particles are exposed on a surface of the catalyst.

9. The catalyst of claim 1, wherein at least 75% by weight of the total weight of the gold particles are within an outer 50% of the volume of the catalyst.

10. The catalyst of claim 1, wherein the support has an average diameter of at least 1.5 mm.

11. The catalyst of claim 1, wherein the support comprises an oxide of silicon and an oxide of titanium.

12. A method for preparing methyl methacrylate from methacrolein and methanol; said method comprising contacting in a reactor a mixture comprising methacrolein, methanol and oxygen in the presence of a catalyst according to claim 1.
